(19) United States
(12) Patent Application Publication
(10) Pub. No.: US 2015/0012007 A1
(43) Pub. Date: Jan. 8, 2015

(54) INTRODUCER-BASED BLEED DETECTION TECHNIQUE

(71) Applicant: Saranas, Inc., Houston, TX (US)

(72) Inventor: Christopher Alexander Arevalos, Houston, TX (US)

(21) Appl. No.: 14/323,371

(22) Filed: Jul. 3, 2014

Related U.S. Application Data
(60) Provisional application No. 61/842,763, filed on Jul. 3, 2013.

(51) Int. Cl. A61M 25/06

(52) U.S. Cl. CPC A61M 25/0662; A61M 2025/0681; A61M 2025/0687
USPC 606/108

(57) ABSTRACT

An embodiment of a system is disclosed including an introducer usable to insert a catheter into a blood vessel of a patient. The introducer further includes a hollow sheath to receive the catheter when inserting the catheter into the blood vessel. In addition, the system includes a hollow sleeve to be disposed around the sheath. Further, the system includes a first electrode provided on the sleeve. Still further, the system includes a measuring device to be coupled to the first electrode and to a second electrode to be coupled to the first electrode through the patient's tissue. The measuring device is configured to detect a bleed in the patient's tissue by computing an impedance or a conduction velocity between the first electrode on the sleeve and the second electrode.

### CROSS-REFERENCE TO RELATED APPLICATIONS

This application claims priority to U.S. Prov. App. No. 61/842,763 filed Jul. 3, 2013 and entitled “Introducer-Based Bleed Detection Technique,” which is incorporated herein by reference in its entirety for all purposes.

### BACKGROUND

1. Field of the Disclosure

This invention relates generally to the field of medical devices. More specifically, the invention relates to a method and device using impedance for the detection of fluid (e.g., blood) bleeding such as pericardial effusion, retroperitoneal effusion, etc.

2. Background Information

Radiofrequency ablation (RF ablation) or other invasive cardiac procedures which involve operation within the cardiac chambers, coronary arteries or the heart's venous anatomy have saved many lives. These procedures often involve percutaneous access into the cardiac chambers or epicardial arterial or venous vessels. Catheter, pacing lead, sheath, or other types of device manipulations frequently are performed as key parts of these procedures. An example of this includes balloon angioplasty or stent placement. Often, catheter access to the femoral artery is needed to enable access to the heart or elsewhere in the body.

A rare but potentially dangerous complication of these and similar procedures is the inadvertent perforation of a cardiac chamber or an epicardial vessel. Retroperitoneal bleeding, arteriovenous fistula, pseudoaneurysms, and hematoma formation is also possible at the site of the insertion of the catheter into the femoral or other artery or vein. Perforations of a cardiac chamber or an epicardial vessel may lead to accumulation of blood (or other fluids) in the pericardial space or sac. This condition is referred to pericardial effusion. Cardiac tamponade is the patho-physiologic state wherein accumulation of blood or other fluid in the pericardial space or sac leads to impaired filling of the heart and a secondary decrease in cardiac output and consequential hemodynamic derangement. It is not unusual in clinical procedures for the onset of perforation to be heralded by the onset of hemodynamic derangements such as drop in blood pressure. In such cases it is frequently only at that time that the presence of a perforation is recognized. Much time may have elapsed between the creation of a perforation and the subsequent accumulation of enough blood or fluid to create a hemodynamically-significant pericardial effusion or tamponade. Of critical clinical significance is that early detection of such perforation may allow the operator to implement interventions (for example discontinuation of peri-operative anticoagulation) that would mitigate the untoward consequences of pericardial effusion.

Retroperitoneal bleeding, arteriovenous fistulae, or hematomas may lead to hemotoma formation, pain, blood loss, shock, or death. Their detection frequently only occurs after hypotension or other symptoms are noted, and there may be no other signs associated with the bleeding. As in the case of a pericardial effusion, prompt recognition offers the opportunity for potentially lifesaving intervention. Another frequent complication of such procedures involves development of blood clots (“thrombosis”) within the body of the sheath. These clots may travel (“embolize”) via the circulation and lead to necrosis or ischemia of tissue subserved by these blood vessels.

It follows that a method and device which could more rapidly detect the presence of pericardial or retroperitoneal bleeding, aretriovenous fistula, or hematoma, prior to the onset of symptoms, is highly desirable. Rapid detection of such bleeding or fluid accumulation can lead to more timely management—such as aborting the procedure or reversal of the patient's anticoagulation response during such cardiac procedures.

### BRIEF SUMMARY

Some embodiment are directed to a system including an introducer usable to insert a catheter into a blood vessel of a patient, the introducer including a hollow sheath to receive the catheter when inserting the catheter into the blood vessel. In addition, the system includes a hollow sleeve to be disposed around the sheath. Further, the system includes a first electrode provided on the sleeve. Still further, the system includes a measuring device to be coupled to the first electrode and to a second electrode to be coupled to the first electrode through the patient's tissue. The measuring device is configured to detect a bleed in the patient's tissue by computing an impedance or a conduction velocity between the first electrode on the sleeve and the second electrode.

Other embodiments are directed to a system including a sheath configured to introduce a catheter into a blood vessel of a patient. In addition, the system includes a hollow sleeve configured to be disposed about at least a portion of the sheath, wherein the sleeve comprises an electrically conductive material. Further, the system includes an electrode electrically to be attached to the patient's body and to be electrically coupled to the sleeve's electrically conductive material via the patient's tissue. Still further, the system includes a measuring device electrically to be coupled to the sleeve and to the electrode. The measuring device is configured to detect a bleed in the patient's tissue by measuring the impedance or conduction velocity between the sleeve and the electrode.

Still other embodiments are directed to a system including a sheath configured to introduce a catheter into a blood vessel surrounded at least partially by tissue. In addition, the system includes a hollow sleeve disposable around the sheath, wherein the hollow sheath is insertable within the tissue. Further, the system includes a plurality of electrodes disposed on the sleeve. Still further, the system includes a measurement device coupled to the electrodes, wherein the measurement device is configured to fix one of a current or voltage across a first of the electrodes and measure the other of the current or voltage across a second of the electrodes. The first electrode and the second electrode are electrically coupled to one another through the tissue when the sleeve is disposed within the tissue. The measurement device is configured to compute one of an impedance and a conduction velocity between the first electrode and the second electrode and determine if there is blood within the tissue based on the computed impedance or conduction velocity.

### BRIEF DESCRIPTION OF THE DRAWINGS

FIG. 1 is a schematic perspective view of an introducer sheath with an electrode usable to determine impedance for the detection of bleeding in accordance with various embodiments.

FIG. 2 shows a cross-sectional view of the sheath with a partial ring electrode on an exterior surface in accordance with various embodiments.

FIG. 3 shows a cross-sectional view of the sheath with a complete ring electrode on the exterior surface in accordance with various embodiments.

FIG. 4 shows a cross-sectional view of the sheath with an electrode embedded in the material of the sheath. in accordance with various embodiments

FIG. 5 shows a cross-sectional view of the sheath with an electrode on an interior surface of the sheath in accordance with various embodiments.

FIG. 6 shows a schematic view of the introducer sheath of FIG. 1 being inserted into a blood vessel of a person and connected to an impedance measuring apparatus in accordance with various embodiments.

FIG. 7 shows a method in accordance with various embodiments.

FIG. 8 shows a box diagram of an impedance measuring apparatus in accordance with various embodiments.

FIG. 9 illustrates various impedance thresholds stored in the impedance measuring apparatus.

FIG. 10 depicts an illustrative method of calibrating the impedance measuring apparatus of FIG. 8.

FIG. 11 is a schematic perspective view of another introducer sheath with an electrode usable to determine impedance for the detection of bleeding in accordance with various embodiments.

FIG. 12 shows a box diagram of an impedance assessment unit in accordance with various embodiments.

FIG. 13 shows a schematic view of the introducer sheath of FIG. 11 being inserted into a blood vessel of a person and connected to an impedance measuring apparatus in accordance with various embodiments.

FIG. 14 is a schematic perspective view of an bleed detecting sleeve disposable on an introducer sheath and usable to determine impedance for the detection of bleeding in accordance with various embodiments.

FIG. 15 is a schematic view of the bleed detecting sheath of FIG. 14 disposed around an introducer sheath and inserted into the tissue of a person.

### NOTATION AND NOMENCLATURE

In the following discussion and in the claims, the term “fluid” is defined to include blood and other types of body fluids or gases that may bleed or leak from a vessel or organ. All references to an impedance measurement being made encompasses any of the variations described herein as performed by the combination of the impedance assessment unit and an external apparatus. In addition, in the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection, or through an indirect connection via other devices, components, and connections. In addition, as used herein, the terms “axial” and “axially” generally mean along or parallel to a central axis (e.g., central axis of a body or a port), while the terms “radial” and “radially” generally mean perpendicular to the central axis. For instance, an axial distance refers to a distance measured along or parallel to the central axis, and a radial distance means a distance measured perpendicular to the central axis.

### DETAILED DESCRIPTION

In accordance with exemplary embodiments disclosed herein, systems and methods are disclosed herein that involve real-time assessment of resistance or impedance to an electrical signal (current or voltage). Accumulation of sufficient fluid or blood in such areas as the pericardial space leads to changes in both the direct current (DC) resistance and/or the complex impedance to alternating current (AC) current flow. A change in either the resistance or the complex impedance signals will occur due to fluid accumulation in the space through which the electrical current travels. Embodiments disclosed herein also use conduction time between two vectors as another variable which may be analyzed. Various embodiments are described herein for measuring impedance to detect fluid bleeding. Impedance may be computed by injecting a known current (DC or AC) and measuring the resulting voltage, or imposing a known voltage across the electrodes and measuring the resulting current. The ratio of voltage to current determines impedance.

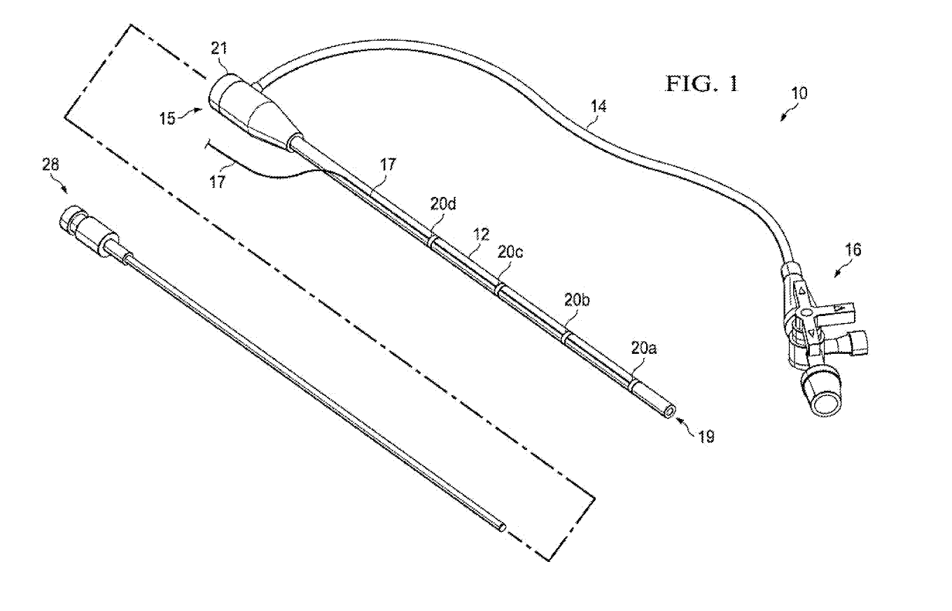

In accordance with one such embodiment, FIG. 1 illustrates an introducer 10 usable to insert a catheter into a blood vessel (vein or artery). The introducer comprises a hollow sheath 12 having a distal end 19 that is insertable into a blood vessel of a person. The blood vessel may be an artery or a vein. In at least one application, the blood vessel is the femoral artery, but other blood vessels may be used as well. In the illustrative embodiment of FIG. 1, multiple electrodes 20a, 20b, 20c, and 20d are provided on the sheath 12. Such electrodes can be provided at any of a variety of locations along the sheath 12. As will be explained below, the electrodes are usable to measure impedance of the person so as to detect bleeding (e.g., retroperitoneal bleeding).

Impedance between pairs of electrodes 20a-20d within the sheath 12 can also be measured to assess the presence of such phenomenona as clots within the sheath 12. In this embodiment, the system can be based on using only one pair of the electrodes 20a-20d such that the injected current and the detected voltage are from one pair of the electrodes 20a-20d, or on multiple pairs of the electrodes 20a-20d such that the injected current and the detected voltage are two separate pairs of the electrodes 20a-20d. For example, one pair of the electrodes 20a-20d is used to inject current and another pair of the electrodes 20a-20d is used to measure the resulting voltage to thereby assess impedance, or vice versa (a known voltage is imposed on pair of the electrodes 20a-20d and current is measured). Although two different pairs of the electrodes 20a-20d are used, an electrode may be common to both pairs. Other configurations utilizing multiple electrodes are also feasible embodiments.

The sheath 12 may be coupled to a hub 15 which may incorporate a hemostasis valve 21 from which a side arm 14 may extend that allows the sheath to be used to administer fluids and/or drugs. A valve 16 is provided on the opposing end of the side arm 14. The introducer 10 also includes a dilator 28 that is insertable into the hollow sheath 12. The dilator 28 and sheath 12 function to permit a catheter to be inserted into the blood vessel. Independently from the preceding features, the sheath 12 may also include other features to facilitate simple “peel-away” removal without disturbing a catheter having been passed the lumen of the sheath 12. Referring still to FIG. 1, electrical conductors 17 (e.g., wires) extend along at least part of the sheath 12 from the electrodes 20a-20d and can be connected to an external device (i.e., a device external to the person/patient that receives the sheath 12). The conductors 17 preferably include at least one insulated conductor for each electrode 20a-20d. The conductors 17 are usable to conduct signals between at least one electrode 20 on the sheath 12 and the external device for impedance measurements.

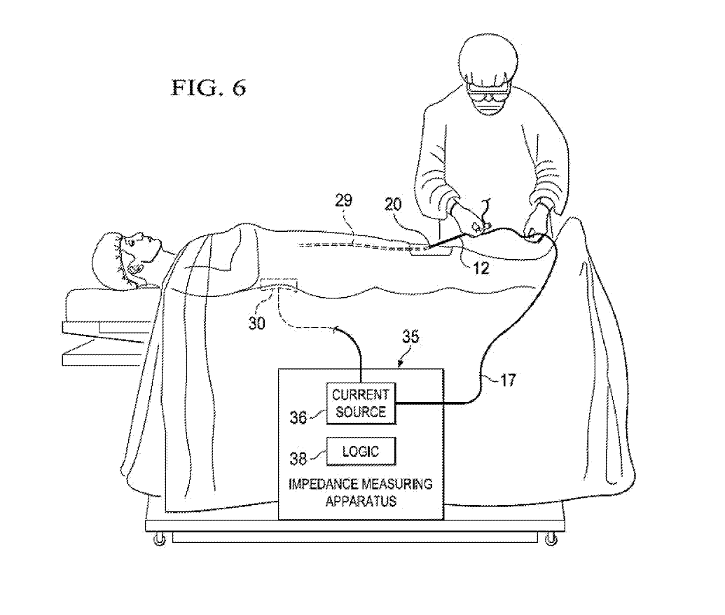

In FIG. 1, four electrodes 20a-20d are shown on the sheath 12, but in other embodiments, more or less than four electrodes may be provided on the sheath 12. Alternately only one electrode (e.g., one of the electrodes 20a-20d) may be present on the sheath 12, with another electrode used in the impedance measurements being located apart from the sheath (e.g., an external electrode as illustrated in FIG. 6). Impedances between any individual pair of the electrodes 20a-20d may be measured.

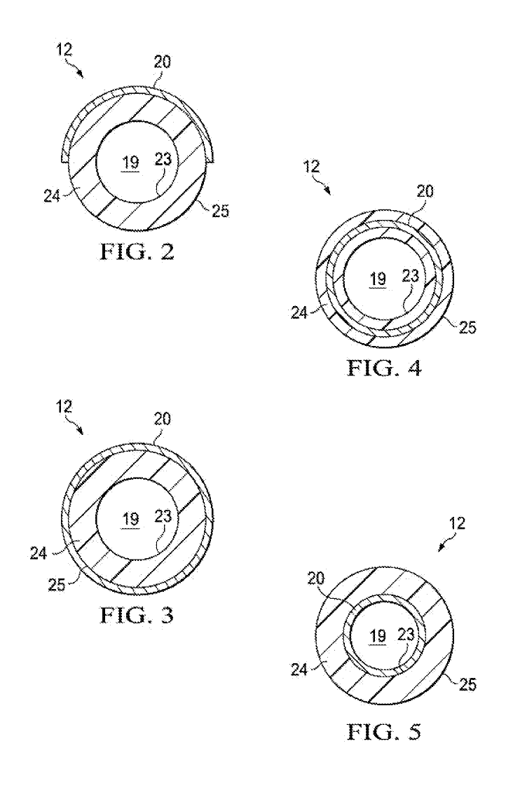

FIGS. 2-5 illustrate various embodiments of the electrodes 20a-20d. Each figure shows a cross-sectional view of the sheath 12 facing distal end 19. Referring first to FIG. 2, sheath 12 comprises material 24 formed as a tubular member and comprising an inner surface 23 and an outer surface 25. In FIG. 1, the electrode 20 (which may be any one of the electrodes 20a-20d) comprises a partial ring electrode disposed about a portion of the perimeter of the outer surface 25. In some embodiments, the electrode 20 is adhered (e.g. via glue) to the outer surface 25. In other embodiments, the electrode 20 covers more than 50% of the perimeter of the outer surface 25 and is retained (e.g., clamped) in place like a bracelet.

FIG. 3 illustrates an embodiment of the electrode 20 in which the electrode is a complete ring electrode (i.e., completely surrounds the outer surface 25 of the sheath 12).

In FIGS. 2 and 3, the electrode 20 is provided on the outer surface 25 of the sheath 12. In FIG. 4, the electrode 20 is embedded within the material 24 of the sheath 12 in which case the sheath materials (or at least the segments of the sheath material between the electrodes) must be conductive of electrical signals in the range employed. Furthermore, for the purposes of detection of clot, impedance or conduction (for detecting bleeding) between these electrodes 20 may be measured. In FIG. 5, the electrode 20 is provided on the inner surface 23 of the sheath 12 and thus within the inner hollow portion of the sheath 12.

In some embodiments, the electrode 20 is located on the sheath 12 so that the electrode 20 will be inside the blood vessel once the sheath 12 is inserted into the vessel. In other embodiments, the electrode 20 may be provided on the sheath 12 at the proximal end outside the blood vessel (and perhaps even outside the person's body). In such embodiments, the electrode 20 preferably is provided on the inner surface of the sheath 12 (similar to that shown in FIG. 5). Normally, the sheath 12 is filled with body fluid (e.g., blood).

FIG. 6 illustrates a person 13 lying supine with the sheath 12 inserted into a blood vessel 29. The conductors 17 from the electrodes 20 (e.g., electrodes 20a-20d) are connected to an impedance measuring apparatus 35. The impedance measuring apparatus 35 comprises a current source 36 and logic 38. The current source 36 may be part of the logic 38 if desired. One or more additional electrodes 30 is also connected to the impedance measuring apparatus 35. Such an additional electrode 30 is also herein referred to as the non-sheath electrode 30 or the non-sheath electrodes 30. The current source 36 injects an electrical current through one of the electrodes 20 or 30. The current then passes through the person's tissues, into the other electrode 20, 30 and back to the current source 36. The injected current may comprise a series of pulses or a sustained current. The amplitude of the current may be in the sub-physiological range, such as 1 milliamp. If a pulse train is used, the pulse width may be 0.2 milliseconds or less and have a frequency of between 5,000 and 500,000 Hz or higher. Additionally or alternatively, the current source 36 may inject current at a plurality of frequencies simultaneously. The plurality of frequencies may, for example, be five frequencies, which may be 5 KHz, 10 KHz, 50 KHz, 100 KHz, and 500 KHz.

The current source 36 or logic 38 measures the voltage across the electrodes 20, 30 resulting from the current, and calculates the ratio of the voltage to current to compute impedance. The impedance is altered in the presence of bleeding and thus can be correlated to bleeding such as retroperitoneal bleeding. The presence of the bleeding may also be referred to herein as an internal bleeding complication (IBC). The device may also calculate the conduction time between the electrodes 20, 30. Bleeding will also alter the conduction time between tissues. Alternatively, rather than a current source (e.g., current source 36), a voltage source can be used to impose a specified voltage on the electrodes 20, 30 and the resulting current level is measured to compute impedance.

The logic 38 may comprise an IBC algorithm to detect an IBC that may occur during the introduction of the catheter into the blood vessel 29. The algorithm may be represented by two main segments—a calibration segment and a measurement or monitoring segment. The IBC algorithm, or simply the algorithm, may be used by the logic 38 to detect the IBC based on the measured impedance data and to set an alarm upon detecting the IBC event. The algorithm may begin processing the impedance data at insertion of the sheath 12 into the person 13 and it may continue to process until the sheath 12 is removed so that there is continuous monitoring of the person 13.

The calibration segment of the algorithm may be implemented by the logic 38 during the first few seconds, as much as 20 seconds, after the sheath 12 is inserted into the person 13 and the impedance measuring apparatus 35 is enabled. The calibration segment may establish a baseline impedance value and function for the person 13 for each of the plurality of frequencies, which may then be used by the monitoring segment to determine if any bleeding has occurred. The algorithm's calibration segment may first take a couple of impedance measurements at each of the plurality of frequencies then take their average to establish a baseline impedance value. This baseline impedance value may then be used to normalize all subsequent impedance measurements.

After the baseline impedance value has been established, the logic 38 may begin to generate a time dependent line of the impedance measurements by adding the absolute value of the impedance change from measurement to measurement. The line shows that the impedance of the person 13 drifts, and this drift may be due to polarization of the human tissue and inherent noise of the system. The impedance measuring apparatus 35 may implement a low noise filter to filter out some of the noise, but the noise may continue to affect the drift in the measurement. As the logic 38 continues to generate the line from the impedance measurements (again, this is done for each of the plurality of frequencies), the algorithm may fit the line with a representative function. The fit may be used later by the monitoring segment of the algorithm.

Lastly, the algorithm may calculate a distribution and a standard deviation for the baseline impedance across the plurality of the frequencies. The distribution may show the spread in the impedance measurements across the plurality of frequencies and the standard deviation may be used by the algorithm to establish a threshold for the impedance measurements. The threshold may be used by the logic 38 to determine when to turn on an alarm alerting the physician of a potential IBC event. The threshold may be set at a multiple of the standard deviation of the distribution of the baseline impedance values, for example, three standard deviations may be the threshold.

The calibration segment of the algorithm may be processed by the logic 38 and may be completed in only a few seconds of being enabled. In presence of an IBC event, the calibration segment may be completed before any bleeding has had time to grow to a level that may be detected by the logic 38.

The logic 38, after completing the calibration segment, may transition into the monitoring segment of the algorithm. The monitoring segment may continue to process and analyze subsequent impedance measurements and compare them to the baseline distribution to determine the presence of an IBC. At each of the subsequent impedance measurements after the calibration segment has finished the algorithm may first normalize the impedance value for each of the plurality of frequencies by the baseline impedance value for the corresponding frequency. The normalized impedance values for each of the plurality of frequencies may then be reduced by the fit line established during calibration. A distribution, average and standard deviation for the resulting values may then be computed. The distribution and/or the standard deviation may then be compared to (e.g., subtracted from) the threshold to determine whether or not an IBC event has occurred. For example, if the distribution of the subsequent impedance values has changed by more than three standard deviations away from the baseline distribution, then the logic 38 may set off an alarm. On the other hand, if the distributions are within the threshold of one another, then the logic 38 continues to run the algorithm.

The comparison of the distribution may also include computing the average impedance across the plurality of frequencies for each subsequent impedance measurement then comparing that average to the threshold, which may be three times the baseline standard deviation. If the impedance value for any of the plurality of frequencies changes more than the three standard deviation threshold, then the logic 38 sounds the alarm. The comparison of each frequency to the threshold separately may be calculated since differences in impedance changes at the various frequencies being different due to variations in the person's tissue. Some frequencies may be more sensitive to the bleeding therefore detecting it sooner.

The monitoring segment of the IBC algorithm may be performed on impedance measurements on virtually any time scale, but may have a lower limit determined by the logic 38. The monitoring analysis steps of the algorithm may be set by the attending physician to be performed every second, 5 seconds, 10 seconds, minute, etc. Alternatively, the frequency of the analysis may be dynamically altered during operation so to perform the analysis more or less often. The frequency at which the monitoring segment of the algorithm is run may be selected through an interface of the impedance measuring apparatus 35. The selection mechanism may be a knob selecting a time interval, or a soft input that allows a user to set a time interval by entering a number of seconds, or a series of switches associated with a different time either in seconds or minutes.

The non-sheath electrodes 30 may be located at any of a variety of locations. The illustrative embodiment of FIG. 6 shows the non-sheath electrode 30 attached to the skin on the back of person 13 as a patch electrode. In other embodiments for detecting retroperitoneal bleeding, the non-sheath electrode 30 can be attached to a urinary catheter, a rectal temperature probe, an electrosurgical grounding pad, or a patch on a lateral aspect of the back as desired, separately or in combination.

In accordance with at least some embodiments, the sheath 12 may comprise two or more electrodes 20. Another pair of electrodes (e.g., electrode 30) may be attached to the patient's skin (e.g. back, abdomen) as noted above. One pair comprising one of the sheath electrodes 20 and one of the skin electrodes 30 is used to inject current through one electrode 20, 30 and the other of the pair of electrodes (i.e., the other sheath electrode and skin electrode) is used to measure the resulting voltage for the impedance computation.

In another embodiment, the sheath 12 may include four electrodes 20 as shown in FIG. 1. Two electrodes 20 are placed distally near the distal tip 19 and two are placed proximally of the shaft of the sheath. A current is sent through one distal electrode 20, through the patient's body to one of the proximal electrodes 20 (the direction of current flow can be in the opposite direction as well). The remaining pair of electrodes 20 measures the voltage for the impedance computation.

In some embodiments, each possible pair of electrodes is used to send/receive current with the remaining electrodes used to measure voltage for an impedance computation.

In some embodiments, each possible pair of electrodes is used to send/receive current with the remaining electrodes used to measure voltage for an impedance calculation. The system may cycle through each such electrode pair combination.

In yet other embodiments, the sheath 12 may not have any electrodes 20. Instead, multiple electrodes (e.g., 5 or more) are placed on the patient's abdomen near the tip of electrode-less sheath. As before, each possible electrode combination is cycled through the process of sending the current, conducting the current, measuring the voltage, and computing the impedance.

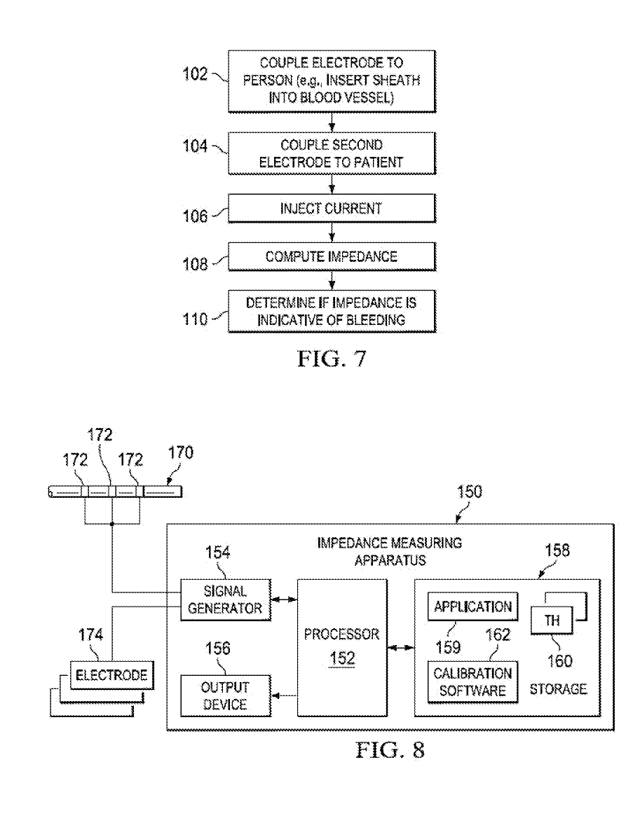

FIG. 7 shows an illustrative method. At 102, an electrode (e.g., electrodes 20, 30) is coupled to the person (e.g., person 13). In the embodiments of FIGS. 1-6, one or more electrodes are located on an introducer sheath 12 and coupled to a person as the sheath 12 is inserted into a blood vessel. At 104, one or more non-sheath electrodes 30 are also coupled to the person 13 (e.g., back electrode 30 shown in FIG. 6). At 106, upon a user activating a control on the impedance measuring device to begin the impedance measuring activity, the impedance measuring apparatus 35 injects current and, at 108, computes the impedance (e.g., measures the voltage and computes the ratio of voltage to current).

At 110, the impedance measuring apparatus 35 determines if the impedance is indicative of bleeding. In some embodiments, the logic 38 of the impedance (or conduction time) measuring apparatus 35 compares the computed impedance to a predetermined threshold, derived threshold based on baseline measurements at the onset of the procedure, otherwise defined acceptable range. The logic 38 determines that bleeding has occurred if the computed impedance or conduction time is outside of the acceptable range for the threshold as previously defined. If bleeding has been detected, the logic 38 may alert a user via an audible and/or visual indicator.

In some embodiments, the impedance measuring apparatus 35 injects a known current and measures the resulting voltage to determine impedance. In other embodiments, the impedance measuring apparatus 35 applies a known voltage to the electrodes and measures the resulting current to determine impedance.

It may be desirable to leave the sheath 12 in place in the person's blood vessel (e.g., vessel 29) following the completion of the medical procedure (e.g., RF ablation) for which the sheath 12 was used in the first place. It is possible that bleeding (e.g., retroperitoneal bleeding) will begin after the completion of the medical procedure. With the sheath 12 still in place, impedance measurements can be made via the impedance measuring apparatus 35 to detect post-medical procedure completion onset of bleeding. A user of the impedance measuring apparatus 35 can activate a control (e.g., press a button) on the impedance measuring apparatus 35 to activate an impedance/bleed monitoring.

Besides retroperitoneal bleeding, arteriovenous fistulae, or hematomas, other types of internal bleeding may occur as well. For example, during a catheterization procedure of a patient's heart or surrounding blood vessel(s), bleeding can occur into the pericardial space. In accordance with various embodiments, a catheter includes one or more electrodes, at least one of which is used to make impedance measurements as described above to detect bleeding such as pericardial effusion. In another embodiment the tip of the catheter or electrode may be located on any guide wire used during coronary intervention (a wire over which a coronary stent or angioplasty apparatus may be advanced is always utilized during such procedures). In this embodiment, the guide wire is or contains an electrode. In such a situation the impedance between the tip of the wire and any second electrode as described elsewhere (such as a skin patch electrode) can be utilized. In another embodiment a distal and proximal electrode (relative to the location of coronary blockage which is to be angioplastied or stented) within the same wire may be used to assess progression of clot formation or perforation and effusion.

FIG. 8 illustrates an embodiment of an impedance measuring apparatus 150 usable to measure impedance and detect bleeding. Any of the attributes described below for impedance measuring apparatus 150 can apply to impedance measuring apparatus 35 of FIG. 6 as well. The impedance measuring apparatus 150 comprises a processor 152, a detector 153, a signal generator 154, an output device 156 and storage 158. The storage 158 comprises volatile memory (e.g., random access memory), non-volatile storage (e.g., read only memory, hard disk drive, Flash storage, etc.), or combinations thereof. The storage 158 comprises an application 159 usable to perform impedance measurements and detect bleeding as described herein and calibration software 162. Both applications 159 and 162 are executed by processor 152. Storage 158 is also used to store one or more impedance thresholds 160. The impedance measuring apparatus 150 comprises logic which includes any combination or all of the processor 152, signal generator 154, and storage 158 (and associated applications and thresholds stored thereon).

Electrodes 172 are provided on a catheter 170 and electrically coupled to the signal generator 154. One or more additional electrodes 174 may also be provided and coupled to signal generator 154. Under control of the processor 152 (via execution of application 159), the signal generator 154 selects one pair of electrodes 172, 174, applies a known current using a plurality of frequencies, as discussed above, to one of the electrodes in the selected pair and receives the current via the electrode. The detector 153 determines the resulting voltage across a selected pair of electrodes 172, 174, which may be the same pair or a different pair of electrodes from that pair used to apply the voltage, and provides the voltage measurement to the processor 152. The detector 153 may comprise an analog-to-digital converter to convert the voltage measurement to digital form for the processor 152. Both the current and voltage values are provided to the processor 152 which then computes the impedance (ratio of voltage to current), or conduction time and compares the computed impedance or conduction time to a corresponding threshold to determine if bleeding has occurred. A pair of electrodes can be selected coupling two of the electrodes 172, 174 to the signal generator (via a switching device). The signal generator 154 can select two electrodes from among electrodes 172 on the catheter 170, two electrodes from among electrodes 174, or one electrode each from electrode sets 172 and 174.

If two electrodes 172 are selected on the catheter 170, the impedance measuring apparatus 150 can detect a blood clot within the catheter 170 by measuring the impedance between the two catheter electrodes 172. The same is true with respect to the embodiment of FIG. 1. The sheath 12, in some embodiments, comprises more than one electrode 20. The impedance measuring apparatus 35 measures the impedance between the electrodes 20 on the sheath 12 to detect blood clots that may form within the sheath 12.

The catheter 170 can be inserted into any of a variety of veins or arteries. In one embodiment, the catheter 170 is inserted into the femoral artery (for detection, for example, of retroperitoneal effusion), the heart or coronary vasculature such as the coronary sinus (for detection of pericardial effusion), or other blood vessels or anatomic structures. The coronary sinus is an epicardial vein through which venous drainage of coronary circulation occurs. It is on the inferior surface of the left atrium. More distally this structure turns into the great cardiac vein or any of its other tributaries.

The electrodes 174 may be located at any of variety of sites. An electrode 174, for example, may be located on the person's esophagus, on the person's skin, or on the person's heart. Moreover, impedance can be measured for detecting bleeding between, for example, the coronary sinus and skin, coronary sinus and esophagus, skin and skin (e.g., patient's front and back), heart and coronary sinus, heart and esophagus, two sites on the same catheter, two sites on the same sheath, two sites on the same vein and femoral artery to skin.

As explained herein, more than two electrodes can be used for measuring impedance. Impedance can be measured between any pair of electrodes and such an impedance measurement represents a vector. For example, in a three-electrode system (first, second, and third electrodes), there are three possible impedance vectors including the impedance between the first and second electrodes, the impedance between the first and third electrodes, and the impedance between the second and third electrodes. The number of vectors increases disproportionately with increasing numbers of electrodes. The physical location of the various electrodes may be useful to detect bleeding in different locations. For example, bleeding may occur between the first and second electrodes, but the fluid (e.g., blood) may not be present between the second and third electrodes. Thus, in this example, the impedance vector associated with the first and second electrodes may be indicative of the bleed, but not so the impedance vector associated with the second and third electrodes or possibly the first and third electrodes. Moreover, more than two electrodes provides an enhanced ability to detect bleeding in different locations than might be possible in a two-electrode only system.

In some embodiments, the computed impedance may be resistance while in other embodiments, the computed impedance is complex having both amplitude and phase components. In other embodiments the computed variable is conduction velocity. Further, the impedance measuring apparatus 150 (or impedance measuring apparatus 35 in FIG. 6) determines and stores an impedance threshold for each impedance vector. Two or more of the various impedance thresholds may be the same or the impedance thresholds may all be different. Each impedance threshold may be an amplitude only value (resistance) or, in the case of complex impedance, comprise an amplitude value and a phase value.

The calibration software 162 may implement the calibration segment of the IBC algorithm discussed above in conjunction with logic 38. The calibration software 162 may use the impedance values calculated by the processor 152 to establish the baseline impedance values, the baseline impedance distribution and standard deviation along with the fit line. The distribution and standard deviation may be used to determine an impedance threshold for detecting an IBC event. The establishment and calculation of all the calibration values may be performed using several of the first measurements before a bleed, if one had occurred, may be severe enough to be detectable.

The application 159 may contain the monitoring segment of the IBC algorithm discussed above in relation to the logic 38 of FIG. 6. The application 159 may use the impedance values calculated by the processor 152. The application 159 may normalize the subsequent impedance values using the baseline impedance values. As subsequent measurements are made, the application 159 may compare the impedance values for each of the plurality of frequencies to the threshold established by the calibration software 162. If a subsequent impedance value for one of the plurality of frequencies exceeds the threshold, then the impedance measuring apparatus 150 may direct the output device 156 to alert a physician to a possible IBC event.

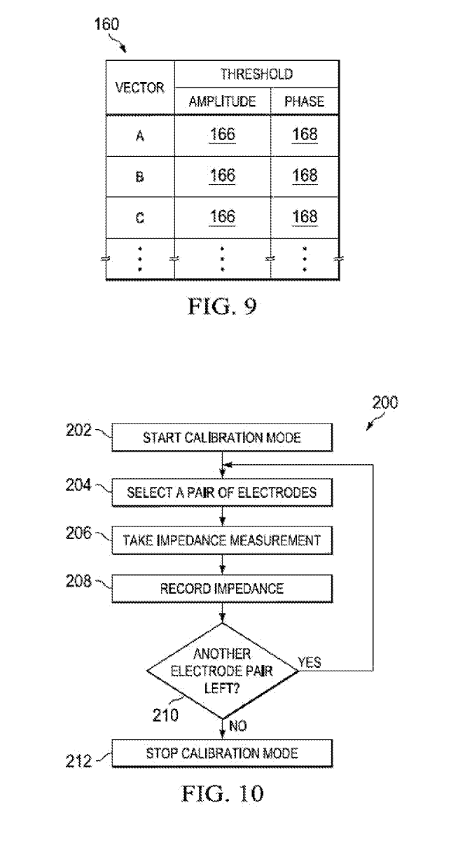

FIG. 9 illustrates the thresholds 160 as a table comprising one or more vectors A, B, C, etc. Each vector represents a pair of electrodes. For each vector, there is an amplitude threshold value 166 and/or a phase threshold value 168. In some embodiments, the impedance measuring apparatus detects the presence of bleeding if either of the amplitude or phase of the computed impedance for a given vector exceeds its corresponding amplitude or phase threshold value, respectively. In other embodiments, the impedance measuring apparatus detects a bleed only if both the computed amplitude and phase exceed their corresponding threshold counterparts. The threshold vales of amplitude and phase may be derived in a variety of ways, one of which may be baseline measurements at the beginning of the procedure for each individual patient as explained below.

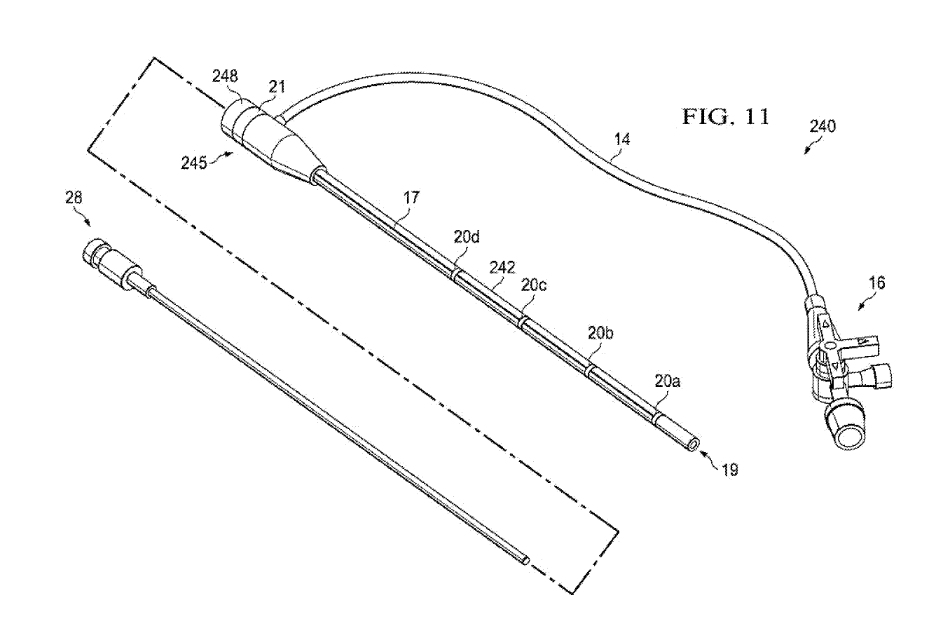

FIG. 10 illustrates a method 200 for calibrating the impedance measuring apparatus (35 or 150) for the various thresholds. In some embodiments, the impedance measuring apparatus comprises a calibration mode that can be initiated by a user of the impedance measuring apparatus (e.g., by pressing a button). The processor 152 of the impedance measuring apparatus 150 executes the calibration software 162 (impedance measuring apparatus 35 may also have similar software to be executed by a processor). FIG. 10 is a method performed by the processor upon executing the calibration software 162. The calibration mode is performed preferably before the medical procedure [e.g., coronary angiography (after the wire has been placed through the blockage but before angioplasty) or electrophysiology study (after catheters have been placed in the coronary sinus but before delivery of radiofrequency ablation)] begins.

The calibration mode begins at 202. A pair of electrodes is selected at 204 and at 206 and 208, an impedance measurement is taken and the computed impedance is recorded (e.g., stored in storage 158) (as amplitude and/or phase values). Preferably, the impedance measurement for a selected pair of electrodes is taken over the course of several breaths by the patient. The impedance computed for the selected impedance vector will vary during a respiratory cycle. By taking the impedance measurement over the course of several breaths (e.g., 10 seconds), the impedance measuring apparatus can account for the normal variations in impedance. The threshold (amplitude or phase) may be computed as an average during the recording period or may be set as the peak value detected (or a value slightly higher (e.g., 5% higher) than the peak). At 210, the impedance measuring apparatus determines whether there is an additional impedance vector for which a threshold is to be determined. If there is, control loops back to step 204 at which such an electrode pair is selected. If not more electrode pairs are to be selected, than the calibration mode stops at 212. This calibration process may take several minutes. The same calibration variables may be measured for conduction velocities.

Once the calibration process is completed, the medical procedure (which might result in bleeding or clot formation) can begin. Any bleeding will be detected as a change in impedance above deviating from an impedance threshold (e.g., an increase above the threshold or decrease below the threshold).

The impedance measuring techniques described herein to detect bleeding are also usable to detect a hemothorax. In this application, electrode locations would include the anterior chest and posterior chest walls, the esophagus at the level near the heart, the trachea, as well as numerous intravascular and intra-cardiac and intra-coronary locations. The electrodes may be on catheters or wires.

With regards to conduction velocity, the logic (e.g., that contained in the measuring devices described herein) assesses the conduction time between the onset of the electrical impulse in the first (transmitting) electrode and second (receiving) electrode. These electrodes are identical to the electrodes described in embodiments disclosed herein. The electrical output is in the same range with regards to frequency and amplitude. The measured variable, however, is the difference (delta) in time (usually milliseconds) between onset of stimulus (electrical output) in the transmitting electrode and sensing of that impulse (electrical sensing) in the receiving electrode. Conduction velocity is heterogeneous with variations in tissue characteristic. As fluid develops, the conduction velocity between the transmitting and receiving electrode will also change. This will be noted as a deviation from a baseline value (similar to the impedance values/thresholds described herein). An alert will then be issued. The various embodiments of apparatus and methods described above can also be used to measure conduction velocity and use conduction velocity to determine thickening of the heart and the presence of fluid bleeding.

In accordance with some embodiments, the sheath 12 includes a wireless transceiver that is able to wirelessly transmit impedance values or impedance-related values to an external apparatus rather than via a wired connection as shown in FIG. 6. The embodiment of introducer 240 in FIG. 11 is similar in some ways to introducer 10 of FIG. 1. Introducer 240 includes sheath 242 which includes multiple electrodes 20a-20d as well a hub 245. Hub 245 includes an impedance assessment unit 248.

As will be explained below, the impedance assessment unit 248 connects to the electrodes via conductors 17 and is used during an impedance measurement. The impedance assessment unit 248 may include a power source. In one embodiment, the impedance assessment unit 248 sets a predetermined current or voltage for one pair of electrodes and measures the resulting voltage or current from another electrode pair. The impedance assessment unit 248 may also include a wireless transmitter to transmit the measured voltage/current to the external apparatus which in turn computes impedance based on the received, measured voltage/current and a prior knowledge of the predetermined current/voltage set by the impedance assessment unit 248 using the algorithm discussed in association with logic 38 of FIG. 6. Alternatively, the impedance assessment unit 248 may also wirelessly transmit the current/voltage it set to the external apparatus. Further still, the impedance assessment unit 248 may itself compute the impedance value and wirelessly transmit the computed impedance value to the external apparatus which may then implement the IBC algorithm discussed above. These and other embodiments are discussed below.

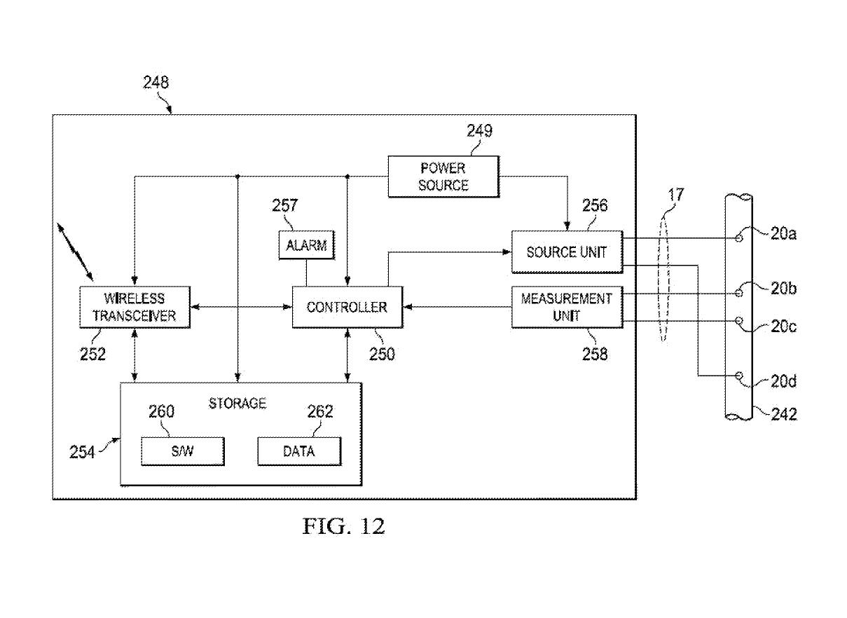

FIG. 12 shows a block diagram of the impedance assessment unit 248. As shown in the example of FIG. 12, the impedance assessment unit 248 includes a power source 259, a controller 250, a wireless transceiver 252, storage 254, a source unit 256, an alarm 257, and a measurement unit 258.

The power source 249 may comprise a battery (disposable or rechargeable), a charged capacitor, a wireless power receiver, or other sources of electrical power. The power source 249 provides electrical power to the controller 250, wireless transceiver 252, storage 254 and source unit 256.

The controller 250 executes software 260 provided on storage 254. The controller 250, upon executing software 260, provides the impedance assessment unit 248 with some or all of the functionality described herein. The storage 254 may comprise volatile storage (e.g., random access memory), non-volatile storage (e.g., flash storage, read only memory, etc.), or combinations of both volatile and non-volatile storage. Data 262 consumed or produced by the software can also be stored on storage 254. For example, measured current or voltage values, computed impedance values, etc. can be stored on storage 254 pending wireless transmission through the wireless transceiver 252 to an external apparatus.

The wireless transceiver 252 may be implemented in accordance with any suitable wireless protocol such as BLUETOOTH, WiFi, etc. The transceiver 252 may be capable of transmitting only, or may be capable of transmitting and receiving. The controller 250 causes the wireless transceiver 252 to transmit values indicative of impedance (current, voltage) or impedance values themselves. The transceiver 252 may be a bi-directional device to permit outgoing transmissions of data, as well as receive incoming commands from an external apparatus. For example, an external apparatus may send a command to the controller 250 via the wireless transceiver 252 to command the impedance assessment unit 248 to initiate a process by which impedance is determined, or to transmit previously stored data (e.g., current, voltage, and/or impedance).

The source unit 256 receives power from the power source 249 and generates a current or voltage under control by the controller 250. The source unit 256 may generate a predetermined current or voltage, and is broadly referred to as a source unit to indicate either or both possibilities. The source unit 256 is connected to a pair of electrodes (electrodes 20a and 20d in the example of FIG. 12), As a current source, the source unit 256 injects a current through one of the electrodes 20a-20d and receives the return current through the other of the electrodes 20a-20d.

The measurement unit 258 measures the resulting voltage or current. That is, if the source unit 256 injects a predetermined current into the patient, the measurement unit 258 measures the resulting voltage. If the source unit 256 imposes a predetermined voltage across electrodes 20a-20d, the measurement unit 258 measures the resulting current. In either case, the measurement unit 258 provides the measured electrical parameter to the controller 250.

The controller 250 thus knows the magnitude of the predetermined current or voltage generated by the source unit 256 and the magnitude of the measured voltage or current from the measurement unit 258. As such, the controller 250 can compute impedance, and does so as the ratio of voltage to current and transmit the computed impedance to the external apparatus. However, as noted above, the controller 250 may not compute impedance and instead transmit the measured electrical parameter (voltage or current) to the external apparatus for the external apparatus to compute impedance. The external apparatus may or may not know what predetermined current or voltage was set by the source unit 256. If the external apparatus does know the magnitude of the source unit's current/voltage, that value need not be (but of course can be) transmitted to the external apparatus. If the external apparatus is not aware of the source unit's current/voltage magnitude, the controller 250 preferably transmits both the measured voltage/current from the measurement unit 258 and the source unit's predetermined current/voltage.

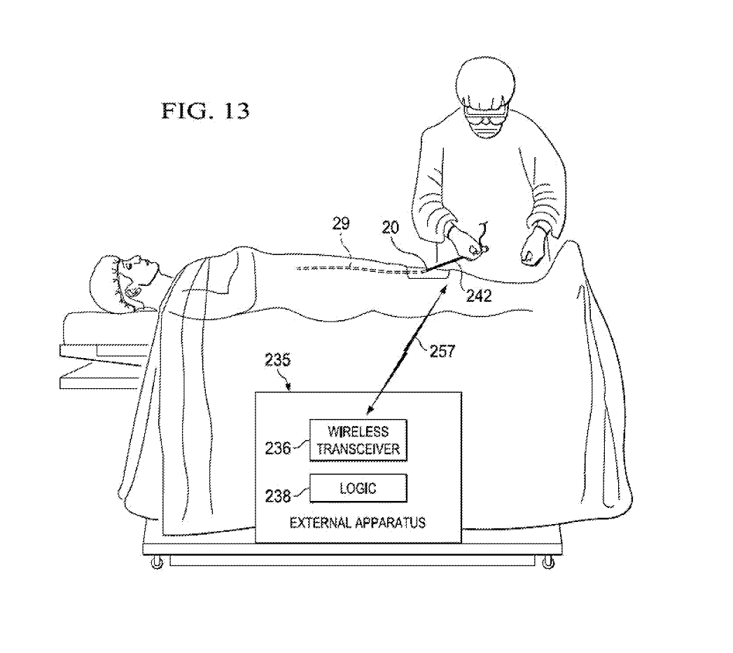

FIG. 13 illustrates an application of the use of the impedance assessment unit 248. Sheath 242 containing impedance assessment unit 248 is inserted into a patient's blood vessel as shown and a wireless communication link 257 is established to an external apparatus 235. The external apparatus 235 may contain a corresponding wireless transceiver 236 as well as logic 238. The logic 238 may implement the IBC algorithm discussed above, or alternatively, the impedance assessment unit 248 may implement the IBC algorithm. The external apparatus 235 may be a computer (desktop, laptop, notebook, etc.), a smart phone, or any other type of device capable wirelessly interacting with the impedance assessment unit 248 of sheath 242. In some embodiments, the external apparatus 235 is, or is built into, a bedside monitor.

The external apparatus 235 may also comprise a means of receiving inputs from a user (not shown) so the user may configure the impedance assessment unit 248 to perform monitoring events at predetermined times or, alternatively, to set a desired time frame (e.g., every 5 seconds) for the IBC algorithm to perform monitoring analysis. For example, the input may be a knob used to select one of several periods (e.g., 1 second, 5 seconds, 10 seconds, 1 minute, etc.) or the input device may be a computer drop down menu with the same time frames. Alternatively, the external apparatus 235 may contain a manual trigger so that the analysis is performed whenever the trigger is activated. Further, the input may allow a user to enter a specific time for performing the analysis, e.g., every 10 minutes.

Regardless of whether the impedance assessment unit 248 computes impedance or transmits the necessary data for the external apparatus 235 to compute the impedance, the computed impedance may be resistance based on DC current/voltage. In other embodiments, AC current/voltage is used and complex impedance is computed as a magnitude and a phase. AC currents/voltages have an associated frequency and impedance measurements can be made at any one or more of multiple different frequencies. All references to an impedance measurement being made encompass any of the variations described herein as performed by the combination of the impedance assessment unit 248 and an external apparatus 235.

Impedance measurements made at certain frequencies may provide more useful information than at other frequencies. At certain frequencies, it may be difficult to detect a bleed, where as other frequencies, bleed detection is easier. Further, the particular frequenc(ies) useful to detect a bleed may vary from patient to patient. Accordingly, a calibration is performed at the beginning of a procedure using a sheath as described above. The calibration may include the calibration segment of the IBC algorithm discussed above in relation to the logic 38 of FIG. 6. The calibration may entail performing multiple impedance measurements at various frequencies. In some implementations, the range of acceptable frequencies is from 1000 Hz to 500 KHz, although a different frequency range may be acceptable as well. Within the frequency range multiple discrete frequencies are chosen to make the impedance measurement. For example, 10 KHz may be chosen as well as 1000 Hz, and 100 KHz.

The source unit 256 may be capable of injecting an AC current (or generating an AC voltage) at various frequencies as commanded by the controller 250. The controller 250 preferably is configured (e.g., by way of software 260) to initiate multiple impedance measurements at various frequencies during the calibration process. Each measured electrical parameter (e.g., voltage) may be stored in data 262 in storage 254 and mapped to the frequency of the source signal (e.g., current) that caused the measured voltage to occur. Thus, multiple AC voltages (or current) may be stored in storage 254, one voltage (or current) corresponding to each AC current (or voltage) frequency. The measured parameters may be kept in storage 254 and/or wirelessly transmitted to the external apparatus 235.

The calibration process may be initiated in any suitable manner. For example, a wireless command to initiate the calibration process may be transmitted from the external apparatus 235 to the impedance assessment unit 248. Alternatively, impedance assessment unit 248 may have a user input control (e.g., a button, switch, etc.) that a user can activate to initiate the calibration process. Further still and in the case in which the power source is a battery, an electrically insulative strip may prevent at least one of the battery's contacts from connecting the to the rest of the impedance assessment unit 248 circuitry. Removal of the strip may cause the controller 250 to initialize and start the calibration process.

Then, at predetermined time periods (e.g., once per minute) after calibration, the controller 250 initiates additional impedance measurements to be made. The controller 250 may implement the monitoring segment of the IBC algorithm discussed above and that may be located in the software 260 of the storage 254 if the external apparatus 235 does not implement the IBC algorithm. At the expiration of each such time period, the controller 250 may also cause multiple impedance measurements to be initiated at the same frequencies used during the calibration process. After computing the impedance values at the various frequencies (whether the impedance assessment unit or the external apparatus makes the computation as explained above), a comparison is made between each such impedance value and a previously computed threshold based on the impedance distribution of the baseline calibration values. A determination is made as to whether the difference, as an absolute value, between the impedance value and the previously computed impedance value (e.g., calibration impedance value) is greater than a predetermined threshold (e.g., greater than three times the baseline standard deviation). An impedance difference greater than the threshold is an indicator of a bleed. Another way to make the comparison is compute a ratio of the current impedance value to the previously computed impedance value and then compare the ratio to a predetermined range. A ratio being outside the range is an indicator of a bleed. Bleeds may be easier to detect at certain frequencies rather than others for certain patients and thus the probability is higher that an actual bleed will be detected if multiple frequencies are used.

The process of taking impedance measurements and comparing to a previous impedance value (e.g., calibration impedance values) is repeated at the expiration of each subsequent time period. Additionally or alternatively, the impedance assessment unit 248 may be triggered manually to initiate an impedance measurement. The user can activate the user control noted above, if such a user control is provided, or the external apparatus 235 may wirelessly transmit a command to cause the controller 250 to initiate a new impedance measurement.

Referring again to FIG. 12, in embodiments in which the impedance assessment unit 248 computes impedance, the controller 250 may activate the alarm 257 if a potential bleed is detected. The alarm may be a visual indicator such as a light emitting diode (LED), an audible indicator such as a piezo-electric device, or both.

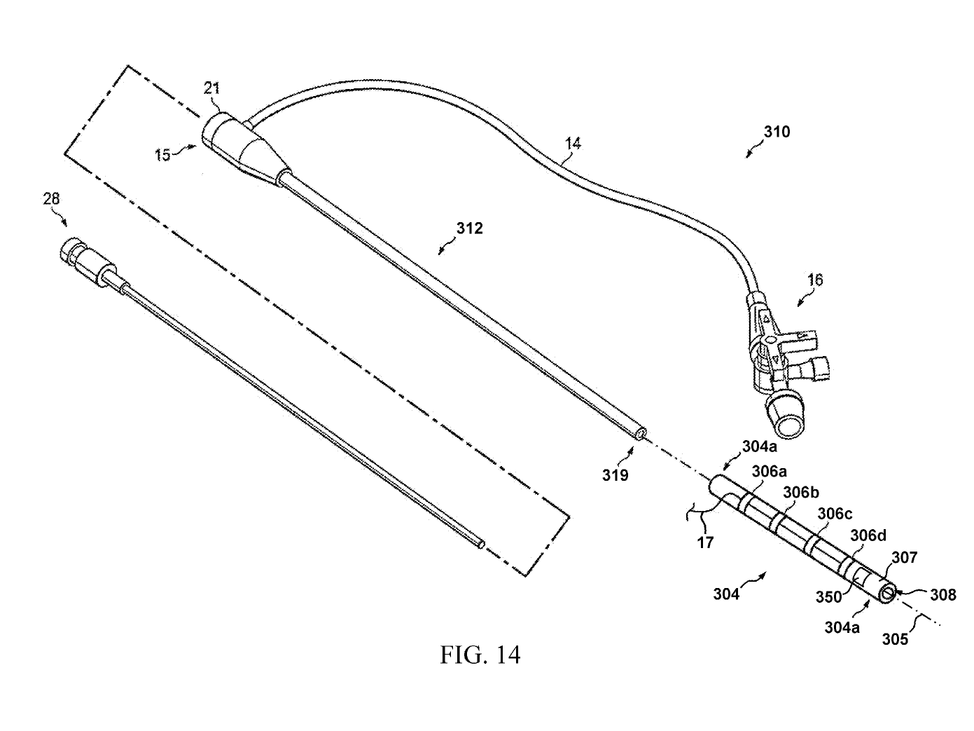

FIG. 14 depicts another introducer 310 usable to insert a catheter into a blood vessel (vein or artery) that is further configured to perform bleed detection operations in accordance with the principles disclosed herein. The introducer 310 comprises a hollow sheath 312 that includes a distal end 319 and is substantially the same as sheaths 12, 242, shown in FIGS. 1, 11, respectively, except that no electrodes (e.g., electrodes 20a-20d) are included on the outer surface of sheath 312. In addition, introducer 310 includes hub 15, side arm 14, valve 16, dilator 28, and hemostasis valve 21, each being the same as previously described above for introducer 10.

Further, in this embodiment, introducer 310 includes a bleed detecting cuff or sleeve 304 configured to be disposed around sheath 312. In general, in this embodiment, bleed detection sleeve 304 is an elongate tubular member having a central axis 305 and including a first or proximal end 304a, a second or distal end 304b opposite proximal end 304a, an internal throughbore 308 extending axially between the ends 304a, 304b, and a radially outer surface 307 also extending axially between ends 304a, 304b. Throughbore 308 is sized to allow sheath 312 to be fully received therethrough during operations.

Once disposed around sheath 312, sleeve 304 may be secured to sheath 312 through any suitable fashion while still complying with the principles disclosed herein. For example, in some embodiments, sleeve 304 is secured to sheath 312 through a clip that is attached to proximal end 304a that engages with a corresponding mating receptacle or attachment point on hub 15. In other embodiments, sleeve 304 is secured to sheath 312 through an interference fit (e.g., the inner diameter of the throughbore 308 is slightly smaller than the outer diameter of sheath 312 such that there is radial interference between sheath 312 and sleeve 304 when sheath 312 is installed within throughbore 308). In still other embodiments, sleeve 304 is secured to sheath 312 through an adhesive, such as, for example, a biocompatible adhesive disposed between the outer surface of sheath 312 and the surface defining throughbore 308.

Referring still to FIG. 14, the bleed detection sleeve 304 also includes a plurality of electrodes 306a, 306b, 306c, 306d that are similar in form, function, and operation to the electrodes 20a-20b on sheaths 12, 242 (see FIGS. 1 and 11, respectively), previously described. In particular, electrodes 306a-306d may be disposed along sleeve 304 in the same manner to that described above for sheath 12. For example, any of the mounting arrangements described and shown in FIGS. 2-5 for sheath 12 and electrodes 20a-20d may similarly be employed to mount electrodes 306a-306d on the sleeve 304 (e.g., mounting of electrodes 306a-306d to the radially outer surface 307 of sleeve 304) while still complying with the principles disclosed herein. It should be appreciated however, that while a total of four electrodes 306a-306d are shown disposed along sleeve 304, the number and arrangement of electrodes 306a-306d on sleeve 304 may be greatly varied while still complying with the principles disclosed herein. For example, in some embodiments, more or less than four electrodes 306a-306d may be disposed along sleeve 304. In particular, in some embodiments, only a single electrode 306 is disposed on sleeve. In at least some of these embodiments, one or more non-sleeve, external electrodes (e.g., electrode 30 shown in FIG. 6 and being the same as discussed above), may be attached or placed externally to the body of the patient in the same manner as described above (e.g., disposed on the lower back of the patient). In these embodiments, impedance may be computed between any one or more of the electrodes 306a-306d and the external electrode(s) to determine whether an IBC is occurring in the same manner as described above (e.g., through IBC algorithm of logic 38).

In addition, in this embodiment, each of the electrodes 306a-306d are coupled to one or more external devices by electrical conductors 17 in a manner similar to that described above for electrodes 20a-20d on sheaths 12, 242 (see FIGS. 1 and 11). In particular, in this embodiment, conductors 17 extend along at least a portion of sleeve 304 and preferably include at least one insulated conductor for each of the electrodes 306a-306d. In addition, conductors 17 may be coupled to one or more other devices, such as, for example, impedance measuring apparatuses 35, 150, impedance assessment unit 248, and/or external device 235, such that IBC detection operations with electrodes 306a-306d on sleeve 304 may be carried out in the same manner as described above for sheaths 12, 242 (e.g., see the method of FIG. 7). It should be appreciated that some embodiments of sleeve 304 and/or introducer 310 may allow for wireless communication between electrodes 306a-306d and an external device. Such wireless communication may be carried out in a similar fashion to that described above and shown in FIGS. 11-13 for introducer 240, except that the electrodes are placed on sleeve 304 rather than sheath 242. For example, in some embodiments, an impedance assessment unit 248, configured substantially the same as previously described above, is electrically coupled to each of the electrodes 306a-306d on sleeve 304, and may: (1) receive commands from an external device (e.g., external device 235 shown in FIG. 13), (2) generate a current or voltage that is injected through one or more of the electrodes 306a-306d, (3) measure a voltage or current on another of the electrodes 306a-306d, (4) calculate impedance and/or conduction time, and (5) transmit wireless signals including the calculated impedance and/or conduction time to the external device. The user is then alerted to the occurrence of an IBC based on the calculated impedance or conduction time in a manner consistent with that previously described above. In addition, in some of these embodiments, unit 248 may simply transmit the measured current or voltage to the external unit and may not perform the calculation of impedance and/or conduction time. Further, in these embodiments, impedance assessment unit 248 may be attached to either sleeve 304, sheath 312, or at any other suitable location while still complying with the principles disclosed herein. In addition, in at least some embodiment, introducer 310 may undergo a calibration procedure substantially identical to that described above for sheath 12 (e.g., the calibration segment of the IBC algorithm performed by logic 38).

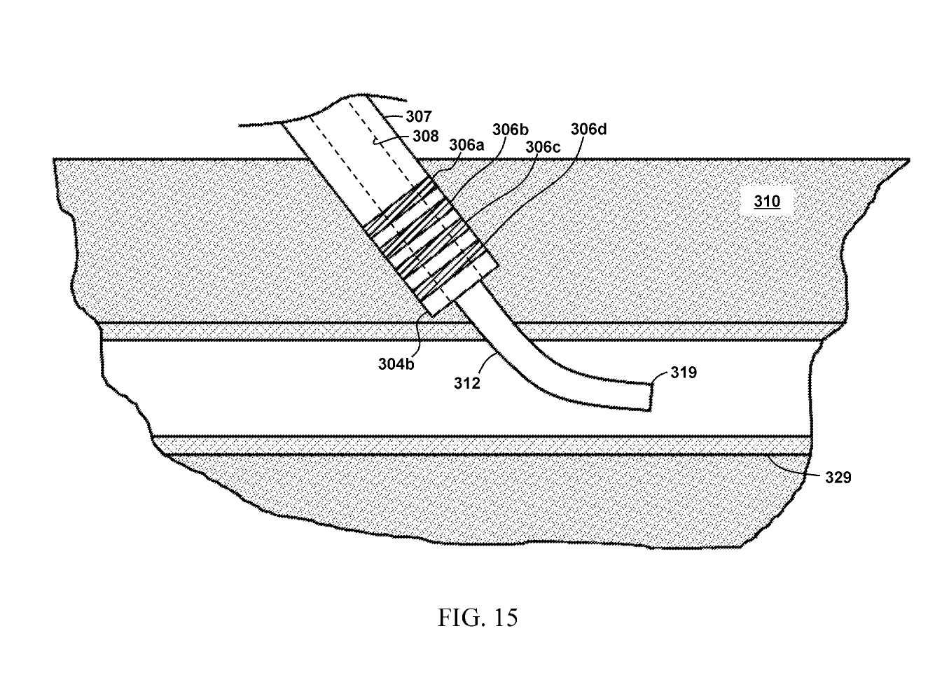

Referring now to FIGS. 14 and 15, the distal end 304b of sleeve 304 and the distal end 319 of sheath 312 may be inserted within the tissue 300 of a patient. Specifically, distal end 304b of sleeve 304 is inserted within tissue 300 but is external to a blood vessel 329 (e.g., vein, artery, etc.), while distal end 319 of sheath 312 is inserted within blood vessel 329 in order to facilitate further insertion of dilator 28 and/or a catheter. However, it should be appreciated that in some embodiments, the distal ends 304a, 319 of sleeve 304 and sheath 312 may both be inserted within vessel 329 while still complying with the principles disclosed herein. As shown in FIG. 14, in some embodiments, sleeve 304 includes a radiopaque marker 350 disposed on outer surface 307 to allow the user to verify and/or determine through an ultrasound image whether sleeve 304 has been properly inserted within tissue 300 and/or vessel 329.

During and/or after the insertion procedure described above sleeve 304, through electrodes 306a-306b may be used to detect an IBC using impedance measurements and analysis in substantially the same manner as described above for sheaths 12, 242. In particular, the bleed detection sleeve 304 may be inserted into tissue 300 so that one or more of the electrodes 306a-306d are in contact therewith in the manner shown in FIG. 15. Thereafter, electrical current may be injected into one or more of the electrodes 306a-306d through conductors 17. The current then passes through the tissue 300 into another one or more of electrodes 306a-306d. An external device(s) (e.g., impedance measuring apparatus 35 shown in FIG. 6), that is coupled to electrodes 306a-306d through conductor 17 and/or a wireless connection, then computes the impedance within tissue 300 based on the measured voltage and/or current across the communicating electrodes 306a-306d to then determine (e.g., through IBC algorithm performed by logic 38) whether an IBC is occurring. In some embodiments, the external device computes the conduction velocity between the communicating electrodes 306aq-306d to determine whether an IBC is occurring in a manner substantially identical to that described above while still complying with the principles disclosed herein.

In some embodiments, sleeve 304 comprises a conductive material that is electrically coupled to one or more of the above described external devices (e.g., impedance measurement apparatus 35) through a wired (e.g., conductor 17) or wireless connection. During operations, electric current is injected into tissue 300 through sleeve 304 directly and is received by an external electrode (e.g., electrode 30 shown in FIG. 6) to then allow the impedance of tissue 300 to be measured in substantially the same manner as previously described. Thus, in at least some of these embodiments, where sleeve 304 comprises a conductive material, no electrodes (e.g., electrodes 306a-306d) are disposed on or in sleeve 304.

In addition, in some embodiments, sleeve 304 is installable by a user on a conventional introducer sheath or other similar device to thereby add the impedance measurement capabilities described herein to such an otherwise conventional device. However, in other embodiments, sleeve 304 is preinstalled onto an introducer sheath (e.g., sheath 312).

While the embodiments of the invention have been shown and described, modifications thereof can be made by one skilled in the art without departing from the spirit and teachings of the invention. The embodiments described and the examples provided herein are exemplary only, and are not intended to be limiting. Many variations and modifications of the invention disclosed herein are possible and are within the scope of the invention. Accordingly, the scope of protection is not limited by the description set out above, but is only limited by the claims which follow, that scope including all equivalents of the subject matter of the claims.

The discussion of a reference in the Background Information is not an admission that it is prior art to the present invention, especially any reference that may have a publication date after the priority date of this application. The disclosures of all patents, patent applications, and publications cited herein are hereby incorporated herein by reference in their entirety, to the extent that they provide exemplary, procedural, or other details supplementary to those set forth herein.

What is claimed is:

1. A system comprising:
an introducer usable to insert a catheter into a blood vessel of a patient, the introducer comprising a hollow sheath to receive the catheter when inserting the catheter into the blood vessel;
a hollow sleeve to be disposed around the sheath;
a first electrode provided on the sleeve; and
a measuring device to be coupled to the first electrode and to a second electrode to be coupled to the first electrode through the patient's tissue, wherein the measuring device is configured to detect a bleed in the patient's tissue by computing an impedance or a conduction velocity between the first electrode on the sleeve and the second electrode.

2. The system of claim 1, wherein the second electrode is provided on the sleeve.

3. The system of claim 1, wherein the second electrode is disposed externally to a body of the patient.

4. The system of claim 1, wherein both the first electrode and the second electrode are external to the blood vessel.

5. The system of claim 1, wherein the measuring device is configured to transmit the computed impedance or conduction time to an external device through a wireless signal.

6. The system of claim 1, wherein the first electrode is coupled to the measuring device through a conductive wire.

7. The system of claim 1, wherein the measuring device detects the bleed by comparing the impedance or the conduction velocity to a threshold value.

8. The system of claim 1, wherein the sleeve has an outer surface, and wherein the sleeve includes a radiopaque marker disposed on the outer surface.

9. A system, comprising:
a sheath configured to introduce a catheter into a blood vessel of a patient;
a hollow sleeve configured to be disposed about at least a portion of the sheath, wherein the sleeve comprises an electrically conductive material;
an electrode electrically to be attached to the patient's body and to be electrically coupled to the sleeve's electrically conductive material via the patient's tissue; and
a measuring device electrically to be coupled to the sleeve and to the electrode, wherein the measuring device is configured to detect a bleed in the patient's tissue by measuring the impedance or conduction velocity between the sleeve and the electrode.

10. The system of claim 9, wherein the measuring device is configured to transmit the computed impedance or conduction time to an external device through a wireless signal.

11. The system of claim 9, wherein at least one of the sleeve and the electrode is to be coupled to the measuring device through a conductive wire.

12. The system of claim 9, wherein the measuring device includes logic that stores a threshold, wherein the logic is to detect a bleed based on a comparison of the computed impedance or conduction velocity to the threshold.

13. A system, comprising:
a sheath configured to introduce a catheter into a blood vessel surrounded at least partially by tissue;
a hollow sleeve disposable around the sheath, wherein the hollow sheath is insertable within the tissue;
a plurality of electrodes disposed on the sleeve; and
a measurement device coupled to the electrodes, wherein the measurement device is configured to fix one of a current or voltage across a first of the electrodes and measure the other of the current or voltage across a second of the electrodes;
wherein the first electrode and the second electrode are electrically coupled to one another through the tissue when the sleeve is disposed within the tissue; and
wherein the measurement device is configured to compute one of an impedance and a conduction velocity between the first electrode and the second electrode and determine if there is blood within the tissue based on the computed impedance or conduction velocity.

14. The system of claim 13, wherein the measuring device includes logic that stores a threshold value, wherein the logic detects a bleed based on a comparison of the computed impedance or conduction velocity to the threshold.

15. The system of claim 13, wherein the measuring device is configured to transmit the computed impedance or conduction time to an external device through a wireless signal.
